(19) United States
(12) Patent Application Publication
(10) Pub. No.: US 2011/0028710 A1
(43) Pub. Date: Feb. 3, 2011

(54) PROCESS FOR THE RECOVERY OF HCL FROM A DILUTE SOLUTION THEREOF AND EXTRACTANT COMPOSITION FOR USE THEREIN

(75) Inventors: Avram Baniel, Jerusalem (IL); Aharon Eyal, Jerusalem (IL)

Correspondence Address: LUCAS & MERCANTI, LLP, 475 PARK AVENUE SOUTH, 15TH FLOOR, NEW YORK, NY, 10016, US

(73) Assignee: HCL CLEANTECH LTD., Tel Aviv (IL)

(21) Appl. No.: 12/935,807

(22) PCT Filed: Apr. 7, 2009
(86) PCT No.: PCT/IL2009/000392

§ 371 Date: Sep. 30, 2010

(30) Foreign Application Priority Data
Apr. 8, 2008 (IL) 190703
Apr. 8, 2008 (IL) 190704
Mar. 3, 2009 (IL) 197379
Apr. 6, 2009 (IL) 198029

(51) Int. Cl. C07H 1/00; C01B 7/01

(52) U.S. Cl. 536/124; 423/488; 252/182.12

(57) ABSTRACT

There is provided a process for the recovery of HCI from a dilute solution thereof, comprising bringing a dilute aqueous HCI solution into contact with a substantially water-immiscible extractant, the extractant comprising an oil soluble amine, which amine is substantially water insoluble both in free and in salt form; an oil soluble weak organic acid having a pKa above 3, which acid is substantially water insoluble both in free and in salt form; and a solvent for the amine and organic acid; whereupon HCI selectively transfers to the extractant to form an HCI-carrying extractant, and treating the HCI-carrying extractant to obtain gaseous HCI. Also provided is the extractant composition.

The present invention relates to a process for the recovery of hydrochloric acid from a dilute solution thereof, as well as to a process for the production of carbohydrates from a polysaccharide by acid hydrolysis with concentrated hydrochloric acid.

The invention is also directed to novel compositions for use in this process.

The term “hydrochloric acid,” as used in the present specification, is intended to denote all forms of hydrochloric acid, including aqueous solutions of hydrogen chloride (HCl) and gaseous phases containing the same. Such acid solutions are broadly present in industrial practice. They are used as reagents (e.g., in regeneration of ion-exchangers) and are formed as by-products or co-products of other processes. In the latter case, the hydrochloric acid obtained is frequently quite dilute, typically 5% HCl to 10% HCl, and needs be reconcentrated to the range of over 20%—desirably to about 30%—to be of commercial viability. Gaseous HCl is particularly attractive. The alternative of neutralization and disposal is inherently costly. In some cases, such dilute HCl solutions are contaminated and reuse requires separation from impurities.

Concentration of hydrochloric acid by distillation is a well-known technology practiced for many years. Its basic drawback is the high cost of the equipment and the inherent large energy consumption. If various impurities are present in the dilute hydrochloric acid, the concentration by distillation needs to be preceded by some separation step to prevent equipment fouling or contamination of the concentrated hydrochloric acid.

In U.S. Pat. No. 4,291,007 by one of the present inventors, there is described and claimed a solvent extraction process for the separation of a strong mineral acid from other species present in an aqueous solution and the recovery thereof under reversible conditions utilizing specific extractants out of the general group of acid-base-couple extractants (hereinafter referred to as an “ABC extractant”) which obviates the consumption of chemicals for regeneration, comprising the steps of:
a) bringing an aqueous solution containing the mineral acid to be separated into contact with a substantially immiscible extractant phase, said extractant phase comprising:
   1) a strong organic acid, which acid is oil-soluble and substantially water-immiscible, in both free and salt forms;
   2) an oil-soluble amine, which amine is substantially water-insoluble, in both free and salt forms; and
   3) a carrier solvent for said organic acid and said amine, wherein the molar ratio of said organic acid to said amine is between about 0.5:2 and 2:0.5,
   whereupon said predetermined mineral acid selectively and reversibly transfers to said extractant phase;
b) separating said two phases; and
c) backwashing said extractant phase with an aqueous system to recover substantially all the mineral acid contained in said extractant phase.

The ABC extractants of that invention were characterized in that the organic acids comprised in them are strong organic acids. The strength of water-soluble acids could be directly determined by the degree of their dissociation in aqueous solution, e.g. as measured by the pH of such solution, which enables calculating the dissociation constant—Ka—or its minus log value pKa. The smaller the pKa the greater is the strength of the acid. For water-insoluble acids, as those in ABC extractants, such direct measurement is impossible. One way of assessing the acidity of such water-insoluble acid is via correlation to water-soluble acids of similar acid function. Considering e.g. HCOOH, (formic acid), CH₃COOH (acetic acid), CH₃CH₂COOH (propionic acid) and CH₃CH₂CH₂COOH (butyric acid), all of which are water soluble. Their pKa values are 3.75, 4.75, 4.87 and 4.81, respectively. The pKa value increased by a full logarithmic unit on adding a CH₃ group to formic acid, but changed very little on adding more CH₂ groups, which have nearly no effect on acid dissociation. Thus, the pKa of a water-insoluble fatty acid with the formula CH₃(CH₂)ₙCOOH is expected to be about 5. (As used herein, the term fatty acid means any acid having the formula CH₃(CH₂)ₙCOOH where n equals 5 or more). The same is expected e.g. for methyl sulfonic acid, ethyl sulfonic acid, propyl sulfonic acid, butyl sulfonic acid and fatty sulfonic acids. Another approach to assessing the pKa of water-insoluble acid is an indirect measurement. On contacting an aqueous solution of the organic acid with an aqueous solution of a salt, e.g. NaCl, an ion-exchange may take place:

HA(org)+NaCl (aq) [symbol] NaA(org)+HCl(aq)

where (org) and (aq) indicate organic phase and aqueous phase respectively and HA is the water-insoluble organic acid. The stronger the organic acid, the more the reaction moves in the right direction and the lower is the pH of the formed aqueous solution. The strong organic acids envisioned for use in the extractant phase of U.S. Pat. No. 4,291,007 were organic acids which may be defined and characterized as follows: When 1 mol of the acid in a 0.2 molar or higher concentration is contacted with an equivalent amount of 1N NaCl, the pH of the sodium chloride solution decreases to below 3.

Especially preferred for use in said invention were strong organic acids selected from the group consisting of aliphatic and aromatic sulfonic acids and alpha-, beta- and gamma-chloro and bromo-substituted carboxylic acids, e.g., hexadecylsulfonic acid, didodecylnaphthalene disulfonic acid, alpha-bromo lauric acid, beta, beta-dichloro decanoic acid and gamma dibromo octanoic acid, etc.

The amines of said invention are preferably primary, secondary and tertiary amines singly or in mixtures and characterized by having at least 10, and preferably at least 14, carbon atoms and at least one hydrophobic group. Such commercially available amines as Primene JM-5, and Primene JM-T (which are primary aliphatic amines in which the nitrogen atom is bonded directly to a tertiary carbon atom) and which commercial amines are sold by Rohm and Haas chemical Co.; Amberlite LA-1 and Amberlite LA-2, which are secondary amines sold by Rohm and Haas; Alamine 336, a tertiary tricaprylyl amine (TCA) and Alamine 304, a tertiary trilaurylamine (TLA), both sold by General Mills, Inc., can be used in the processes of said invention, as well as other well-known and available amines, including, e.g., those secondary and tertiary amines listed in U.S. Pat. No. 3,458,282.

The carrier solvents of said invention could be chosen from a wide range of organic liquids known to persons skilled in the art, which can serve as solvents for said acid-amine active components and which provide for greater ease in handling and extracting control. Said carrier solvents can be unsubstituted or substituted hydrocarbon solvents in which the organic acid and amine are known to be soluble and which are substantially water-insoluble, e.g., kerosene, mineral spirits, naphtha, benzene, xylene, toluene, nitrobenzene, carbon tetrachloride, chloroform, trichloroethylene, etc. Also higher oxygenated compounds such as alcohols, ketones, esters, ethers, etc., that may confer better homogeneity and fluidity and others that are not acids or amines, but which may confer an operationally useful characteristic, can also be included.

In the process of said invention, the essential operating extractant is believed to be the amine, balanced by a substantially equivalent amount of strong organic acid. An excess of acid acts as a modifier of the system, and so does an excess of amine, which obviously will be present as salts of acids present in the system. These modifiers are useful in optimization of the extractant, but are not essential.

Thus, as stated, the molar ratio between the two foregoing active constituents lies between 0.5 to 2 and 2 to 0.5, and preferably between about 0.5 to 1 and 1 to 0.5.

The process as exemplified in said patent was especially useful for use with acids such as nitric acid; however, the process as defined therein wherein the acid is recovered by backwashing is not practical or commercially viable for obtaining concentrated hydrochloric acid from dilute hydrochloric acid.

According to the invention described and claimed in PCT/IL2008/000278, it was surprisingly found that HCl can be distilled out of such an HCl-loaded extractant phase at temperatures below 250° C. without noticeable solvent decomposition.

Thus, said patent describes and claims a process for the recovery of HCl from a dilute solution thereof, comprising:
a) bringing a dilute aqueous HCl solution into contact with a substantially immiscible extractant, said extractant comprising:
   1) an oil soluble amine, which amine is substantially water-insoluble, in both free and salt forms;
   2) an oil soluble organic acid, which acid is substantially water-insoluble, in both free and salt forms; and
   3) a solvent for the amine and organic acid;
      whereupon HCl selectively transfers to said extractant to form an HCl-carrying extractant; and
b) treating said HCl-carrying extractant to obtain gaseous HCl.

Referring to the above-identified PCT application and the teachings and premises thereof, it is to be noted that the distribution of HCl between an extractant and an aqueous phase is adjustable by two simple rules:
a) two ABC extractants composed of the same base and differing in the organic acid that is coupled to it—the extractant that has the stronger organic acid will affect the distribution of HCl in favor of the aqueous phase (decreasing binding with the organic phase) relative to the extractant that has the weaker organic acid; and
b) Two ABC extractants composed of the same organic acid and differing in the base that is coupled to it—the extractant that has the stronger base will affect the distribution of HCl in favor of the solvent phase relative to the extractant that has the weaker base.

These rules are helpful in examining and optimizing the choice of extractant for each particular case that involves the extraction of HCl out of an aqueous phase and recovering it from the extract by back-extraction into H₂O.

It was expected that thermal recovery by “stripping” with an inert gas or vapor will follow similar rules i.e. the stronger of two organic acids that are coupled to the same base will provide for a more effective stripping (i.e. removal from the organic phase)—all else being equal.

As stated hereinbefore with reference to application PCT/IL2008/000278;

(1) “The strong organic acids envisioned for use in the extractant phase of the present invention are organic acids which may be defined and characterized as follows: When 1 mol of the acid in a 0.2 molar or higher concentration is contacted with an equivalent amount of 1N NaCl, the pH of the sodium chloride solution decreases to below 3.

(2) Especially preferred for use in the present invention are organic acids selected from the group consisting of aliphatic and aromatic sulfonic acids and alpha-, beta- and gamma-chloro and bromo-substituted carboxylic acids, e.g., hexadecylsulfonic acid, didodecylnaphthalene disulfonic acid, alpha-bromo lauric acid, beta, beta-dichloro decanoic acid and gamma dibromo octanoic acid, etc.”

In contradistinction to the teachings of said prior art patent, the application and the expectations from the above rules, it was surprisingly observed that weak organic acids, having a pKa above 3 and even very weak organic acids such as fatty acids, can provide for effective stripping of part or the whole of HCl carried in an extractant of which the ABC extractant couples a weak organic acid with an amine.

Stated differently, weak organic acids such as carboxylic acids were not considered of interest in the practice of the invention as described in U.S. Pat. No. 4,291,007 or even as described in more recent application PCT/IL2008/000278, as constituents of ABC extractants or as constituents of extractants for HCl. Such extractants, when equilibrated with an aqueous HCl phase provide for powerful distribution in favor of the extractant, which distribution is only marginally affected by temperature. Stripping i.e. distribution of HCl at higher temperatures in favor of the gas phase was naturally expected to be ineffective in case of weak acids as a component of ABC extractants. Surprisingly it has now been found that that is not the case for fatty acids and similar weak acids, having a pKa above 3 and that effective stripping obtains. Furthermore, the effective extraction of HCl from an aqueous phase, which results in high loading of the extractant, provides for an economically attractive low amount of extractant required per unit of HCl.

Thus according to the present invention, there is now provided a process for the recovery of HCl from a dilute solution thereof, comprising:
a) bringing a dilute aqueous HCl solution into contact with a substantially water-immiscible extractant, said extractant comprising:
   1) an oil soluble amine, which amine is substantially water insoluble both in free and in salt form;
   2) an oil soluble weak organic acid having a pKa above 3, which acid is substantially water insoluble both in free and in salt form; and
   3) a solvent for the amine and organic acid;
   whereupon HCl selectively transfers to said extractant to form an HCl-carrying extractant; and
b) treating said HCl-carrying extractant to obtain gaseous HCl.

Thus, according to the present invention and in contradistinction to the prior art, the organic acids of the extractants of the present embodiment are weak and have pKa above 3, preferably above 3.5, more preferably above 4.0 most preferably above 4.4. According to a preferred embodiment, the pKa of the organic acid for the present invention is determined by that of water-soluble analogs, as explained above. According to another embodiment, the weak organic acids envisioned for use in the extractant phase of the present invention are organic acids, which may be defined and characterized as follows: when 1 mol of the acid in a 0.2 molar or higher concentration in an organic solvent is contacted with an equivalent amount of NaCl in 1N aqueous solution, the pH of the sodium chloride solution is greater than about 4 more preferably greater than about 5.

Thus a weak acid according to the present invention, e.g. carboxylic acids such as lauric acid, when tested according to the above definition, reduces pH only slightly (pH>5).

With regard to the pKa values of the acids mentioned in U.S. Pat. No. 4,291,007 (and PCT/IL2008/000278) as opposed to those envisioned for use in the present invention, said patent refers to
“aromatic sulfonic acids” e.g. Naphtalenesulfonic acid the pKa of which is about zero; and
“alpha-, beta- and gamma-chloro and bromo-substituted carboxylic acids”.

The following table sets forth the pKa values of the analogous water-soluble acids as opposed to the ones estimated for acids suitable for the present invention:

TABLE 1

| Acid | pKa |
|---|---|
| alpha-bromo-butyric | 2.97 |
| 3,6-dichlorophtalic | 1.46 |

Whereas typical values for the (surprisingly observed)
weak acids such as unsubstituted carboxylic acids -

| | |
|---|---|
| Caproic | 4.88 |
| Caprylic | 4.90 |
| Lauric | 4.92 |

Thus the weak organic acids measure 2 or more pKa units higher than the acids previously described and claimed, which corresponds to two orders of magnitude lower acidity.

Thus it was surprisingly observed that weak organic acids, even very weak organic acids such as fatty acids can provide for effective stripping of part or the whole of HCl carried in an extractant of which the ABC extractant couples a weak organic acid with an amine.

The weak organic acids of the present invention are oil soluble and substantially water insoluble both in free and in salt form. As used here, the term “in salt form” means when dissociated. Typically organic acids in salt form have higher solubility in water compared with the same acid in a free form. The solubility in water of the organic acids of the present invention (in both free and salt form) is typically less than 2%, preferably less than 1%, more preferably less than 0.5% and most preferably less than 0.1%. Typically, the organic acids of the present invention have at least 6 carbon atoms, preferably at least 8 carbon atoms, more preferably at least 10 carbon atoms.

The acid function of the organic acid of the present invention is of any type as long as the acid is weak, as defined above. According to a preferred embodiment, the acid function is a carboxylic one. The organic acid may carry a single acid function or multiple such functions. Such multiple functions could be of the same nature, e.g. as in dicarboxylic acids or of a different nature.

According to one embodiment, the organic acid is a fatty acid carrying no substituents. According to other embodiments, the organic acid carries substituents, such as halogen atoms, hydroxyls, carbonyls, etc. In such cases, if the substituent is an electron-pulling one, it is preferably located on a carbon distanced from the carboxylic function (e.g. number 4 or higher). The hydrocarbon chain on the acid could be aromatic or aliphatic, preferably aliphatic. It could be linear or branched, as long as the water-insolubility is maintained.

Example 1 hereinafter is illustrative of manifestation of the contribution of a weak acid to stripping and Example 2 tabulates a number of particular examples that further illustrates the generality and effectiveness of weak organic acids.

The terms “dilute” and “concentrated” as used herein and applied to aqueous phases that contain HCl refer only to the HCl and H₂O. contents in the aqueous phase. Concentrations below 20%/23% HCl, or solutions with H₂O:HCl w/w ratio of about 4 or higher are considered dilute; concentrations above 20%/23% HCl, or solutions with H₂O:HCl w/w ratio of about 3.3 or lower are considered for the purposes of the present specification as being concentrated. The intermediate range of about 20%/23% HCl is commonly referred to as “azeotropic concentration”.

The step of bringing the dilute aqueous HCl solution into contact with the extractant is conducted by methods and in contactors well known in industrial solvent extraction processes. Typically, the extraction is a multistage one, preferably conducted in a counter-current mode of operation. Mixer-settlers, centrifugal contactors and columns are some examples of suitable contactors.

Upon contacting the dilute aqueous solution with the extractant, HCl selectively transfers to the extractant. “Selectively transferred” as used here, means that HCl is preferred by the extractant over other components of the dilute aqueous solution, e.g. water, salts, neutral solutes (e.g. carbohydrates), etc. For example, the HCl/water w/w ratio in the extractant after that contacting is greater than that in the dilute aqueous solution by at least 10 folds, preferably by at least 20 folds, more preferably by at least 50 folds.

Thus the process according to the present invention is capable of recovering HCl practically completely from any aqueous phase whatever the initial concentration; the key usefulness residing in recovering HCl from aqueous phases of initial azeotropic concentrations and lower, e.g. with (HCl/(HCl+H2O)) w/w ration of 20%, 15%, 10% or 5%. Recovery yields (calculated as the w/w ratio between the recovered HCl and the HCl amount in the aqueous solution) of greater than 70%, preferably greater than 80%, more preferably greater than 90%, most preferably greater than 95%. are obtained.

The terms “extractant” and “ABC extractant” are used herein interchangeably.

The amines of the present invention are preferably primary, secondary and tertiary amines singly or in mixtures and characterized by having at least 10, preferably at least 14, carbon atoms and at least one hydrophobic group. Such commercially available amines as Primene JM-5, and Primene JM-T (which are primary aliphatic amines in which the nitrogen atom is bonded directly to a tertiary carbon atom) sold by Rohm and Haas Chemical Co.; Amberlite LA-1 and Amberlite LA-2, which are secondary amines sold by Rohm and Haas; Alamine 336, a tertiary tricaprylyl amine (TCA) and Alamine 304, a tertiary trilaurylamine (TLA), both sold by Cognis, Inc., can be used in the processes of the present invention, as well as other well known and available amines including, e.g., those secondary and tertiary amines listed in U.S. Pat. No. 3,458,282. According to a preferred embodiment, tris(2-ethyl hexyl) amine is used as an amine of the ABC extractant of the present invention.

Similarly with water-insoluble organic acids, directly measuring the basicity of a water-insoluble amine is complicated. Determining basicity via analogy to water-soluble amine could be misleading since that basicity of an amine is strongly dependent on the medium, being much stronger in aqueous solution compared with in an organic one. A known method for an indirect measurement involves equilibrating the amine (or its solution in a hydrocarbon) with an aqueous solution of HCl prepared so that the amount of the acid is one half that of the amine on a mole/mole basis. As used herein, the term equilibrating means contacting, e.g. mixing, until an equilibrium is reached, as determined e.g. by observing no change in the composition of the phases on further contacting. On such equilibrating, a portion of the acid transfers into the organic phase. After equilibrium is reached, the pH of the aqueous solution is determined and referred to as the pH of half neutralization, or pHhn. The stronger the basicity of the amine, the greater is the transfer of the acid into the organic phase and the higher is pHhn. According to various embodiments of the present invention, amines suitable for the extractant of the present invention have a pHhn smaller than 3.5, preferably smaller than 3 and most preferably smaller than 2.5. According to another embodiment of the invention, the pHhn of the amine of the present invention extractant is smaller than the pKa of the organic acid of that extractant.

The above-described method for determining the basicity of water-insoluble amines can also be used in order to determine the basicity of the overall extractant composition (amine+organic acid+solvent). Similarly, the method involves equilibrating the extractant with an aqueous solution of HCl prepared so that the amount of the acid is one half that of the amine content of the extractant on a mole/mole basis. On such equilibrating, a portion of the acid transfers into the extractant. After equilibrium is reached, the pH of the aqueous solution is determined and referred to as the pH of half neutralization, or pHhn of the extractant. According to a preferred embodiment of the invention, the extractant is characterized by a pHhn of less than 3, preferably less than 2.8 and most preferably less than 2.5.

The amines of the present invention are oil soluble and substantially water insoluble both in free and in salt form. As used here, the term “in salt form” means when protonated or positively charged. Typically amines in salt form have higher solubility in water compared with the same amine in a free form. The solubility in water of the amines of the present invention (in both free and salt form) is typically less than 2%, preferably less than 1%, more preferably less than 0.5% and most preferably less than 0.1%.

According to various embodiments of the present invention, the molar ratio between the oil-soluble amine and the weak organic acid lies between 0.1 to 10, preferably between 0.2 and 5 and most preferably between 0.5 and 2. According to an embodiment of the invention, the extractant comprises multiple oil-soluble amines, multiple weak organic acids or both. In such case, the preferred molar ratios are between the total amount of amines and the total amount of weak organic acids. According to a particular embodiment, that ratio is greater than 1, preferably between 1.2 and 4, more preferably between 1.5 and 3.5.

The term “solvent,” as used herein, is intended to refer to any water-immiscible organic liquid in which the acid and amine dissolve. Hydrocarbons, alkanols, esters, etc. having the required immiscibility can be used individually or in admixtures. The terms “water-immiscible” and “water-insoluble” as used here are meant to be synonymous. The solubility in water of the solvent is typically less than 2%, preferably less than 1%, more preferably less than 0.5% and most preferably less than 0.1%

In preferred embodiments of the present invention, the solvent is a hydrocarbon.

To avoid any misunderstanding, it is to be noted that the term “solvent,” as used herein, relates to the third component of the extractant.

A role of the solvent in the extractant of the present invention is to provide for better handling, e.g. to avoid too high a viscosity of the extractant. At the same time, the solvent dilutes the amine and the organic acid in the extractant and reduces thereby the achievable concentration (loading) of extracted HCl in the extractant. The optimal concentrations of the amine (and organic acid) in the solvent is selected by the skilled person according to the concentration of HCl in the dilute aqueous solution, according to the concentration of other components there, according to the temperature of extraction and the temperature of HCl stripping, etc. Typically, the amine concentration in the extractant is between 0.1 and 2.5 mole/Kg, preferably between 0.5 and 2 mole/Kg more preferably between 0.8 and 1.7 mole/Kg.

According to an embodiment, the extract of the present invention comprises two or more oil soluble amines. According to another embodiment, the extract comprises two or more oil soluble weak organic acids. According to still another embodiment, the extractant comprises both multiple amines and multiple organic acids. In such embodiments, the preferred molar ratios between the amines and the organic acids specified above are for the combined concentrations of the amines and the organic acids. The inventors of the present invention have found that in some cases, an extractant comprising a mixture of at least two organic acids, e.g. decanoic and dodecanoic is easier to handle. Thus, according to a preferred embodiment, the extractant of the present invention comprises two water-immiscible weak organic acid or more.

In some cases, when the HCl concentration in the extractant exceeds a given concentration, the extractant splits into two organic phases (so that in instances where an aqueous phase exists, there are a total of three phases). Working with such a two-phase, HCl-comprising, extractant is feasible in normal solvent extraction practices, but might be less convenient than working with a single-phase one. If desired, a person versed in the art can adjust the composition of the extractant to avoid such formation of two organic phases, e.g. by suitable selection of the solvent (or solvent mixture) composition and/or concentration and by selecting the amine(s) and organic acid(s). The inventors have found that, in some cases, using two or more organic acids in the extractant reduces or avoids the formation of such two organic phases.

In preferred embodiments of the present invention, said process further comprises:
c) absorbing the gaseous HCl to provide a hydrochloric acid solution of a higher concentration than that of the HCl in said dilute solution.

Such absorbing is done, according to various embodiments, into water, aqueous solutions, or other solutions. According to one embodiment, the dilute aqueous HCl solution is divided into two portions or more, HCl is extracted from one portion (or more) of the dilute aqueous solution and the gaseous HCl is absorbed in another portion (other portions) to increase HCl concentration there. According to another embodiment, the gaseous HCl is absorbed in an HCl solution formed by other means, e.g. by stripping HCl out of aqueous solutions originally of concentration greater than azeotropic. According to still another embodiment, the gaseous HCl is absorbed in an HCl solution formed in or recycled from another process or another step in the process. Typically, HCl concentration in the provided HCl solution is greater than that in the dilute aqueous solution at least 2 folds, preferably at least 3 folds, more preferably at least 5 folds. According to another embodiment, HCl concentration in the hydrochloric acid solution provided by adsorption is greater than 30%, preferably greater than 35%, more preferably greater than 40% (where concentration is calculated as HCl/(HCL+H2O) w/w).

The process of the present invention comprises a step of treating said HCl-carrying extractant to obtain gaseous HCl. Treating to obtain gaseous HCl here means direct transfer of HCl from the extractant phase into a gaseous phase. Direct transfer here is meant to clearly distinguish the method of the present invention from known methods in which HCl is transferred from the extractant into an aqueous solution (e.g. as in cases of back-washing or back-extraction, as in U.S. Pat. No. 4,291,007), which aqueous solution might be subjected later to distillation. The method of the present invention has important advantages compared with such known methods, including lower water co-distillation, lower energy consumption and a smaller number of operations. Treating the HCl-carrying extractant to obtain gaseous HCl is also referred to herein as stripping. According to a preferred embodiment, said treating comprises heating the HCl-carrying extractant at conditions wherein HCl vapors are formed.

The present invention further provides a process as described hereinabove wherein said heating is at a temperature of up to 250° C., preferably not exceeding 200° C.

In some preferred embodiments of the present invention, said treating comprises introducing a stream of an inert gas for conveying the HCl from said extractant phase. According to various embodiments, said inert gas is selected from a group consisting of nitrogen, CO₂, hydrocarbons, superheated steam and combinations thereof.

In other preferred embodiments of the present invention, said treating comprises a combination of heating and introducing a stream of an inert gas.

In yet another preferred embodiment of the present invention, said treating comprises a combination of heating and sub-atmospheric pressure. The gaseous HCl formed according to the present invention may contain some water vapors. An important advantage of the method of the present invention is that even when HCl recovery is from dilute solution, e.g. HCl concentrations of less than 20%, less than 10% or less than 5%, the moisture content in the formed gaseous product is relatively low, e.g. less than 50%, preferably less than 40%, more preferably less than 30%. Furthermore the HCl/water w/w ratio in the formed gaseous HCl is greater than that ratio in the dilute aqueous phase at least 10 folds, preferably at least 20 folds, more preferably at least 50 folds.

According to various embodiments, the dilute aqueous HCl solution contains impurities. In addition to recovery and concentration, the method of the present invention provides for product purification. Thus, the relative HCl purity with regards to a given impurity (IMP), or to a combination of impurities, as presented by HCl/IMP w/w ratio, is greater in the gaseous HCl compared with that in the dilute aqueous solution by at least 10 folds, preferably by at least 20 folds, more preferably by at least 50 folds.

In another aspect of the present invention, there is provided a process for the production of carbohydrates, comprising:
a) providing a polysaccharide
b) hydrolyzing said polysaccharide in an HCl-containing hydrolysis medium to form a carbohydrate-containing, dilute aqueous HCl solution;
c) bringing said dilute aqueous HCl solution into contact with a substantially water immiscible extractant, said extractant comprising:
   1) an oil-soluble amine, which amine is substantially water-insoluble, in both free and salt forms;
   2) an oil soluble weak organic acid having a pKa above 3, which acid is substantially water insoluble both in free and in salt form; and
   3) a solvent for the amine and organic acid, whereupon HCl selectively transfers to said extractant to form an HCl-carrying extractant and an HCl-depleted carbohydrate-containing solution;
d) treating said HCl-carrying extractant to obtain gaseous HCl; and
e) using said gaseous HCl for hydrolysis of a polysaccharide.

In this aspect of the present invention, said process preferably further comprises a step (f), wherein said gaseous HCl is directly absorbed into a slurry of a comminuted polysaccharide-containing material to generate said HCl-containing hydrolysis medium.

According to various embodiments of this other aspect, the preferred amines for the extractant are selected from the same group of the amines of the previous aspect, the preferred weak organic acids for the extractant are selected from the same group of weak organic acids of the previous aspect, the solvent for the extractant is selected from the same group of the solvents of the previous aspect, the amine to weak acid molar ratio is similar to that of the previous aspect, the amine concentration is similar to that of the previous aspect and combinations thereof.

Any polysaccharide is suitable for the purpose of the present invention, for example a polymer of hexoses, such as glucose and fructose, a polymer of pentoses, such as xylose and arabinose and polymers comprising both hexoses and pentoses. Particularly preferred polysaccharides are cellulose (the main sugar of which is glucose) and hemicellulose (the main sugars of which are xylose and arabinose). Such polysaccharides are the major constituents of several materials, mostly originally from natural sources. Such materials include products of processing wood, e.g. paper (as in recycled paper) and byproducts of such processing, residues of processing crops, such as sugarcane bagasse, straw, corn cobs, etc., by products of processing such crops, such as corn or wheat fibers, wood, grass, energy crops, etc. Many of these and other polysaccharides comprising materials comprise both cellulose and hemicellulose and several other components, the largest of which, in many cases, is lignin. Carbohydrates-containing materials comprising cellulose, hemicellulose and lignin are referred to herein as lignocellulosic materials. Preferably, the polysaccharide-containing material used to provide the polysaccharide according to the present invention is a lignocellulosic material.

The terms sugar, saccharide and carbohydrate are used here interchangeably.

The process of this other aspect comprises the step of hydrolyzing the polysaccharide, e.g. as provided in a polysaccharide-containing material, such as lignocellulosic material, in an HCl-containing hydrolysis medium to form a carbohydrate-containing, dilute aqueous HCl solution. Preferably, said polysaccharide-containing material is comminuted prior to the step of hydrolyzing. Typically, the HCl-containing medium is an aqueous solution comprising HCl, which HCl is highly concentrated, e.g. the HCl/(HCl+H2O) w/w ratio of at least 35%, preferably at least 38%, more preferably at least 40%, most preferably about 42%. Such highly concentrated aqueous solution is sometimes referred to as fuming hydrochloric acid. Such hydrolysis medium may also contain other solutes, e.g. due to being formed via recycle of some process streams. According to a preferred embodiment, said hydrolyzing is conducted at a relatively low temperature, e.g. lower than 50° C., preferably lower than 40° C., more preferably lower than 30° C. Typically, that temperature is higher than the freezing point of the hydrolysis medium, preferably higher than the freezing point of water. Hydrolyzing duration depends on a number of parameters, such as the properties of the polysaccharide-containing material, the size of its comminuted particles and acid concentration. Typically, hydrolyzing duration is between several minutes and several hours, for example, 30 min., 1 h, 2 h or 4 h.

Hydrolyzing of the polysaccharide, e.g. as in a polysaccharide-containing material forms a carbohydrate-containing, dilute aqueous HCl solution. The formed carbohydrate is according to various embodiments of the present invention, a monosaccharide (e.g. glucose, fructose, xylose or arabinose), a disaccharide or an oligosaccharide. Such oligosaccharides, if formed, are soluble in the hydrolysis medium and comprise a relatively small number of carbohydrate monomers (which number is also referred to as degree of polymerization, DP), e.g. less than 10, preferably less than 6, most preferably between 2 and 5.

Preferably, at least 70% of the polysaccharide in said comminuted polysaccharide-containing material is hydrolyzed to carbohydrates. In especially preferred embodiments of the present invention, at least 80% of the polysaccharide is hydrolyzed to carbohydrates, and most preferred, at least 90% of the polysaccharide is hydrolyzed to carbohydrates.

The carbohydrate product of hydrolyzing is typically initially formed in the HCL-concentrated HCl-comprising hydrolysis medium. According to an embodiment of this other aspect, the whole HCl-concentrate, carbohydrate-containing solution, or a portion thereof, is treated for partial removal of HCl therefrom, which partial removal forms said carbohydrate-containing, dilute aqueous HCl solution. Any known method for partial removal is useful here, particularly selective ones in which the formed, removed, HCl is concentrated, i.e. that the amount of water removed with it is relatively small. Such preferred HCl removal over water removal dilutes the HCl concentration in the carbohydrate-containing solution. According to a preferred embodiment, the partial removal of HCl uses HCl distillation or stripping and the HCl concentration in the carbohydrate-containing, dilute aqueous HCl solution is about azeotropic or lower.

According to the process of this other aspect, the carbohydrate-containing, dilute aqueous HCl solution is brought, as such, or after some further treatment, into contact with the above-specified water-immiscibleextractant using methods and apparatus similar to those of the previous aspect, whereby an HCl-depleted carbohydrate-containing solution is formed. In preferred embodiments of the present invention, said HCl-depleted carbohydrate-containing solution provides, as such or after some further treatment, a feedstock for fermentation to generate a fermentation product. Such further treatment comprises, according to various embodiments, pH adjustment, concentration adjustment, partial or substantial removal of soluble inorganic compounds (also referred to as ashes), fractionation into high hexose streams and high pentose stream, addition of nutrients for the microorganisms, e.g. nitrogen sources, and any other treatment required for optimal fermentation.

Preferably, said fermentation product is selected from a group consisting of ethanol, higher alcohols, fatty acids and their esters, fatty alcohols and their esters, lysine, lactic acid and other monomers for the polymer industry. According to a particularly preferred embodiment, the fermentation product is ethanol.

Bringing in contact with the above-specified extractant leads to selective extraction of the majority of the HCl from the carbohydrate-containing, dilute aqueous HCl solution. In said selective extraction, HCl is preferably extracted over water, as in the previous aspect. Furthermore, it is selectively extracted over carbohydrates in said dilute solution. Thus, according to various embodiments, the HCl/carbohydrate w/w ratio in the extractant is greater than that ratio in the carbohydrate-containing dilute HCl solution by at least 50 folds, preferably by at least 100 folds, more preferably by at least 50 folds.

HCl extraction yields are high. Thus, extraction yields (calculated as the w/w ratio between the extracted HCl and the HCl amount in the dilute aqueous solution) are greater than 70%, preferably greater than 80%, more preferably greater than 90%, most preferably greater than 95%. Such bringing in contact reduces HCl concentration in the formed HCl-depleted carbohydrate-containing aqueous solution, e.g., to less than 2%, preferably less than 1%, more preferably less than 0.5%, most preferably less than 0.2%. A characteristic and surprising aspect of the present process is that it combines highly efficient extraction with highly efficient HCl stripping from the HCl-containing extractant (also referred to as extract) generated on that contacting. According to various embodiments, stripping yield (calculated as the w/w ratio between the HCl amount in the gaseous HCl stream and the HCl amount in the extract) is greater than 85%, preferably greater than 90%, more preferably greater than 95%, most preferably greater than 99%. Combining the yield of extracting and the yield of stripping results in the yield of the overall recovery, as calculated by the w/w ratio between the amount of HCl in the gaseous HCl stream and the amount of the HCl in dilute carbohydrate-containing aqueous solution. In some preferred embodiments of the present invention, that ratio is at least 70%. preferably at least 80%, and most preferred, at least 90%.

The process of this aspect further comprises a step of treating said HCl-carrying extractant to obtain gaseous HCl; by means similar to those of the previous aspect and forming gaseous HCl with relatively low water contents similar to those in the previous aspect.

In preferred embodiments of the present invention, said carbohydrate concentration in said HCl-depleted carbohydrate-containing solution is at least 15%. In especially preferred embodiments of the present invention, said carbohydrate concentration in said HCl-depleted carbohydrate-containing solution is at least 20%, and in the most preferred embodiments of the present invention, it is at least 30%.

As indicated above, in some preferred embodiments of the present invention, said polysaccharide is provided in a polysaccharide-containing material, said process further comprising a step of comminuting said material to form a slurry. In preferred embodiments of the present invention, said process further comprises a step (f) wherein said gaseous HCl is directly absorbed into said slurry of a comminuted polysaccharide-containing material to generate said HCl-containing hydrolysis medium. According to some alternative procedures, the gaseous HCl is high in moisture, as e.g. is the case of HCl recovery by distillation from a dilute solution. Absorbing such high-moisture gaseous HCl in such slurry adds water to it. That water, combined with water originally present in the polysaccharide-containing material dilutes the hydrolysis medium, which results in hydrolysis which is too slow. In order to solve that problem, past industrial practice partially or fully dehydrated the polysaccharide-containing material. Such dehydration adds much energy cost. Another related difficulty is that in order to further increase the cost by pulling vacuum on the material, dehydration is conducted at elevated temperatures, e.g. greater than 100° C., sometimes greater than 150° C. At these temperatures some carbohydrates degradation takes place, forming degradation products, which might be inhibitors for the fermenting organisms.

In contradistinction, the gaseous HCl formed according to the process of the present invention is of low moisture, as specified above. Such low moisture minimizes or obviates the need for dehydration of the polysaccharide-containing material prior to forming said slurry. Thus, according to preferred embodiments of the present invention, said provided polysaccharide material is not dried or only partially dried prior to said forming of said slurry.

Preferably said embodiment further comprises steps of providing polysaccharide-comprising material and comminuting it to form said slurry, wherein said provided material has a moisture content of at least 30% or at least 50% and wherein said provided material and said comminuted material are not dried prior to said forming of said slurry or only partially dried.

According to still another embodiment, said polysaccharide is provided in a polysaccharide-containing material and said process further comprises a step of comminuting said material to form a slurry, wherein said provided material and said comminuted material are not exposed to a temperature greater than 100 C.

In yet another preferred embodiment, the present invention is directed to providing a polysaccharide containing material, comminuting it, forming a slurry in a hydrolysis medium, which medium is formed by absorbing gaseous HCl from a previous step and optionally HCl solutions from other operations, hydrolyzing to form carbohydrate in a concentrated HCl solution, separation of part of the HCl from that concentrated solution, preferably by stripping to form a dilute carbohydrate comprising HCl solution, bringing that in contact with the extractant, extracting HCl to form an HCl-depleted carbohydrate solution, optionally used for fermentation, and an HCl-comprising extractant (extract), treating that extract to form (regenerated extractant for reuse) and gaseous HCl and using that gaseous HCl for hydrolyzing polysaccharide, preferably by absorption in a slurry of comminuted polysaccharide-containing material.]

According to an embodiment of the invention the carbohydrate-containing, dilute aqueous HCl solution comprises at least one impurity. The term impurity, as used herein means any soluble component of the solution other than water, HCl and carbohydrates. According to said embodiment, upon bringing said carbohydrate-containing dilute aqueous HCl solution into contact with said extractant, said at least one impurity transfers to said extractant to form an HCl-carrying and impurity-carrying extractant and an HCl-depleted and impurity-depleted carbohydrate-containing solution. The inventors have found that with respect to many impurities, the extractant of the present invention strongly prefers the impurity over the carbohydrate. Thus, according to various embodiments, the w/w ratio between the at least one impurity and the carbohydrates in the HCl-carrying and impurity-carrying extractant is greater than that ratio in the carbohydrate-containing, dilute aqueous HCl solution by at least 5 folds, preferably by at least 20 folds and most preferably by at least 50 folds. As used herein, the term carbohydrates means the total amount of carbohydrates in the solution (e.g. the combined amount of the hexoses and pentoses). According to other embodiments, the w/w ratio between the carbohydrates and said at least one impurity in said HCl-depleted and impurity-depleted carbohydrate-containing solution is greater than that ratio in said carbohydrate-containing, dilute aqueous HCl solution at least 2 folds, at least 5 folds or at least 10 folds. According to an embodiment, said at least one impurity is selected from a group consisting of fermentation inhibitors. According to a related embodiment, said inhibitor is selected from a group consisting of furfural, hydroxymethyl furfural and acetic acid.

In a further aspect of the present invention, there is now provided an organic phase composition comprising:
a. an oil soluble amine having pHhn<3.5, which amine is substantially water insoluble both in free and in salt form;
b. an oil soluble weak organic acid having a pKa>3, which acid is substantially water insoluble both in free and in salt form, and
c. a solvent for the amine and organic acid
   further comprising at least one water-soluble acid selective from the group consisting of HCl, at least one non-volatile acid and combinations thereof.

In preferred embodiments of the present invention said amine carries at least one alkyl chain branched on at least one of an alpha, beta or gamma carbon atom.

In preferred embodiments said water-soluble acid is HCl.

Preferably, said non-volatile acid is selected from a group consisting of H₂SO₄, H₃PO₄, sulfonic acids and combinations thereof.

Preferably said organic phase comprises at least 0.5 mole water-soluble acid per mole of amine.

The present invention is also directed to and provides a composition comprising:
a. an oil soluble amine having pHhn<3.5, which amine is substantially water insoluble both in free and in salt form;
b. an oil soluble weak organic acid having a pKa>3, which acid is substantially water insoluble both in free and in salt form;
c. a solvent for the amine and organic acid;
d. water, and
e. at least one water-soluble acid;

wherein at least one organic phase and at least one aqueous phase exist and wherein said water-soluble acid is distributed between said organic phase and said aqueous phase.

Preferably, said amine carries at least one alkyl chain branched on at least one of an alpha, beta or gamma carbon atom.

In preferred embodiments of the present invention, said water-soluble acid is selected from the group consisting of HCl, at least one non-volatile acid and combinations thereof.

Preferably both said organic phase and said aqueous phase comprise HCl and at least one non-volatile acid.

Preferably, said organic phase comprises at least 0.5 mole water-soluble acid per mole of amine.

In preferred embodiments of the present invention said organic phase further comprises water and the molar ratio between said water and said water-soluble acid in said organic phase is less than 2.

Preferably, said acid distribution has a distribution coefficient greater than 0.3.

In especially preferred embodiments of the present invention, there is provided a composition comprising:
a. an oil soluble amine having pHhn<3.5, which amine is substantially water insoluble both in free and in salt form;
b. an oil soluble weak organic acid having a pKa>3, which acid is substantially water insoluble both in free and in salt form;
c. a solvent for the amine and organic acid; and
d. at least one volatile, water soluble acid,

wherein at least one organic phase and at least one vapor phase exist and wherein said volatile acid is distributed between said organic phase and said vapor phase.

Preferably, said amine carries at least one alkyl chain branched on at least one of an alpha, beta or gamma carbon atom.

Preferably, said volatile acid is HCl.

In preferred embodiments, said composition further comprises, in the organic phase, at least one water-soluble acid selected from a group consisting of HCl, non-volatile acids and combinations thereof.

Preferably, said organic phase comprises at least 0.1 mole water-soluble acid per mole of amine.

Preferably, the concentration of said volatile acid in said organic phase is less than 0.3 mole per mole of amine and the partial vapor pressure of said volatile acid in said vapor phase is greater than 10 mmHg.

The present invention also provides a composition comprising:
a. an oil soluble amine having pHhn<3.5, which amine is substantially water insoluble both in free and in salt form;
b. an oil soluble weak organic acid having a pKa>3, which acid is substantially water insoluble both in free and in salt form;
c. a solvent for the amine and organic acid;
d. water, and
e. at least one volatile, water soluble acid,

wherein at least one organic phase, at least one aqueous phase and at least one vapor phase exist and wherein said volatile acid is distributed between said organic phase and said vapor phase.

Preferably, said amine carries at least one alkyl chain branched on at least one of an alpha, beta or gamma carbon atom.

Preferably, said volatile acid is HCl.

In preferred embodiments of the present invention said volatile acid is distributed between said organic phase, said vapor phase and said aqueous phase.

In preferred embodiments, said composition further comprises, in the organic phase, at least one water-soluble acid selected from a group consisting of HCl, non-volatile acids and combinations thereof.

Preferably, said water-soluble acid is distributed between said organic phase and said aqueous phase.

Preferably, said organic phase comprises at least 0.1 mole water-soluble acid per mole of amine.

Preferably, said organic phase further comprises water and the molar ratio between said water and said water-soluble acid in said organic phase is less than 2.

In preferred embodiments of the present invention, the concentration of said volatile acid in said organic phase is less than 0.3 mole acid per mole of amine and the partial vapor pressure of said volatile acid in said vapor phase is greater than 10 mmHg.

Preferably, said volatile acid is HCl.

Referring now to a further aspect of the present invention, as stated hereinbefore, according to the invention described and claimed in PCT/IL2008/000278, it was surprisingly found that HCl can be distilled out of such an HCl-loaded extractant phase at temperatures below 250° C. without noticeable solvent decomposition.

The recovery of HCl carried by extractant was described therein with respect to two classes of possible stripping-carriers:

1) inert gas, typically N₂; and

2) steam.

Thus in said application, said “treating” comprised heating at a temperature of up to 250° C. and in especially preferred embodiments described therein said “treating” comprised a combination of heating and introducing a stream of inert gas which was described as being preferably N₂ or introducing steam.

As is known, inert gases are effective for stripping—they represent conventional technology and are effective for stripping HCl from HCl-carrying extractant. However, the demands in equipment and operational costs of absorbing the HCl out of a carrier such as N₂ (or CO₂) and recycling the inert carrier present a drawback of this mode of stripping. Furthermore, while water and, generally, aqueous systems are very effective in absorbing the HCl, the N₂ that is thus separated will necessarily carry in it water vapor. The water that is thus recycled decreases the effectiveness where dry HCl is desired.

The use of steam as an inert stripping gas does away with costly recycle since steam condenses to form a liquid water phase and an HCl gas phase. However the liquid phase retains some of the stripped HCl, thereby decreasing overall process efficiency.

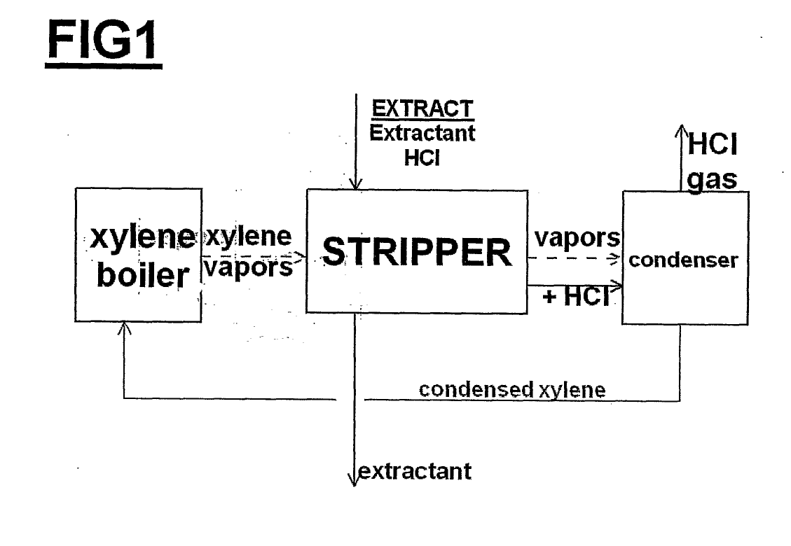

It has now been surprisingly found that the advantages of (1) and of (2) above can be retained with none of their disadvantages by using a hydrocarbon in vapor phase as an inert stripping gas. On cooling the carrier hydrocarbon vapor, it condenses to form a liquid hydrocarbon phase that does not retain any HCl. The HCl is thus recovered fully as a dry HCl phase. Example 3 hereinafter illustrates this finding with a commercial xylene, of 135/145° C. boiling range, as the chosen hydrocarbon vapor and as diagrammatically described with reference to FIG. 1

Thus, according to this further aspect of the present invention, there is now provided a process for the recovery of HCl from a dilute solution thereof, comprising:
a) bringing a dilute aqueous HCl solution into contact with a substantially immiscible extractant, said extractant comprising:
   1) an oil soluble amine which amine is substantially water insoluble both in free and in salt form;
   2) an oil soluble organic acid which acid is substantially water insoluble both in free and in salt form; and
   3) a solvent for the amine and organic acid;
   whereupon HCl selectively transfers to said extractant to form an HCl-carrying extractant; and
b) introducing a stream of an inert stripping gas comprising a hydrocarbon in vapor phase into said HCl-carrying extractant for conveying the HCl from said extractant phase and for obtaining gaseous HCl.

In preferred embodiments of the present invention, said hydrocarbon is selected from the group consisting of aliphatic and aromatic unsubstituted hydrocarbons.

In especially preferred embodiments the hydrocarbon is selected for having, at atmospheric pressure, a boiling point at which it is desired to effect the stripping.

From the above, it will be realized that a first preferred embodiment of the present invention utilizes a hydrocarbon in vapor phase which can be generated outside of the system, used as an inert stripping gas which is then condensed to release HCl and then recycled for further use.

In a further embodiment of the present invention, it is envisioned to generate the hydrocarbon in vapor phase by boiling off some of the “carrier solvent” of the extractant, provided that said hydrocarbon is one that boils at 120 ° C. or higher at atmospheric pressure.

The terms “dilute” and “concentrated” applied to aqueous phases that contain HCl refer only to the HCl and H₂O contained in the aqueous phase. Concentrations below 20%/23% HCl, or H₂O:HCl ratio of about 4 or higher are considered dilute; concentrations above 20%/23% HCl, or H₂O:HCl ratio of about 3.3 or lower are considered concentrated. The intermediate range of about 20%/23% HCl is commonly referred to as “azeotropic concentration”.

Thus the process according to this aspect of the present invention recovers HCl practically completely from any aqueous phase whatever the initial concentration; the key usefulness residing in recovering HCl from aqueous phases of initial azeotropic concentrations and lower.

The terms “extractant” and “ABC extractant” are used herein interchangeably. Preferred for use in this aspect of the present invention are organic acids selected from the group consisting of aliphatic and aromatic sulfonic acids and alpha-, beta- and gamma-chloro and bromo substituted carboxylic acids, e.g., hexadecylsulfonic acid, didodecylnaphthalene disulfonic acid, alpha-bromo lauric acid, beta-, beta-dichloro decanoic acid and gamma dibromo octanoic acid, etc. and organic acids with at least 6, preferably at least 8, and most preferably at least 10, carbon atoms. Especially preferred for use in the present invention are weak organic acids having a pka above 3 as described herein before.

The amines of the present invention are preferably primary, secondary and tertiary amines singly or in mixtures and characterized by having at least 10, preferably at least 14, carbon atoms and at least one hydrophobic group. Such commercially available amines as Primene JM-5, and Primene JM-T (which are primary aliphatic amines in which the nitrogen atom is bonded directly to a tertiary carbon atom) sold by Rohm and Haas Chemical Co.; Amberlite LA-1 and Amberlite LA-2, which are secondary amines sold by Rohm and Haas; Alamine 336, a tertiary tricaprylyl amine (TCA) and Alamine 304, a tertiary trilaurylamine (TLA), both sold by General Mills, Inc., can be used in the processes of the present invention, as well as other well known and available amines including, e.g., those secondary and tertiary amines listed in U.S. Pat. No. 3,458,282.

Furthermore, according to a preferred embodiment, tris(2-ethyl hexyl) amine is used as an amine of the ABC extractant of this aspect of the present invention.

The term “solvent,” as used herein, is intended to refer to any water-immiscible organic liquid in which the acid and amine dissolve. Hydrocarbons, alkanols, esters, etc. having the required immiscibility can be used individually or in admixtures.

In preferred embodiments of the present invention, the solvent is a hydrocarbon.

To avoid any misunderstanding, it is to be noted that the term “solvent,” as used herein, relates to the third component of the extractant.

The term “pH half neutralization (pHhn),” as used herein refers to an aqueous solution, the pH of which is in equilibrium with the extractant carrying HCl at an HCl-to-amine molar/molar ratio of 1:2.

In preferred embodiments of the present invention, said process further comprises:
d) absorbing the gaseous HCl to provide hydrochloric acid of a higher concentration than that of the HCl in said dilute solution.

In another aspect of the present invention, there is provided a process for the production of carbohydrates, comprising:
a) providing a polysaccharide
b) hydrolyzing said polysaccharide in an HCl-containing hydrolysis medium to form a carbohydrate-containing, dilute aqueous HCl solution;
d) bringing said dilute aqueous HCl solution into contact with a substantially immiscible extractant, said extractant comprising:
   1) an oil-soluble amine, which amine is substantially water-insoluble, in both free and salt forms;
   2) an oil-soluble organic acid, which acid is substantially water-insoluble, in both free and salt forms; and
   3) a solvent for the amine and organic acid,
   whereupon HCl selectively transfers to said extractant to form an HCl-carrying extractant and an HCl-depleted hydrocarbon-containing solution;
d) introducing a stream of an inert stripping gas comprising a hydrocarbon in vapor phase into said HCl-carrying extractant for conveying the HCl
   from said extractant phase and for obtaining gaseous HCl.; and
e) using said gaseous HCl for hydrolysis of a polysaccharide.

In this aspect of the present invention, said process preferably further comprises a step (f), wherein said gaseous HCl gas is directly absorbed into a slurry of a comminuted polysaccharide-containing material to generate said HCl-containing hydrolysis medium.

Preferably, said polysaccharide-containing material is a lignocellulosic material

In preferred embodiments of the present invention, said HCl-depleted carbohydrate-containing solution provides a feedstock for fermentation to generate a fermentation product.

Preferably, said fermentation product is ethanol.

While the invention will now be described in connection with certain preferred embodiments in the following examples and with reference to the appended figures so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following examples which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

In the drawings:

FIG. 1 is a schematic flow diagram of recovery of HCl gas using xylene vapors as the stripping inert gas;

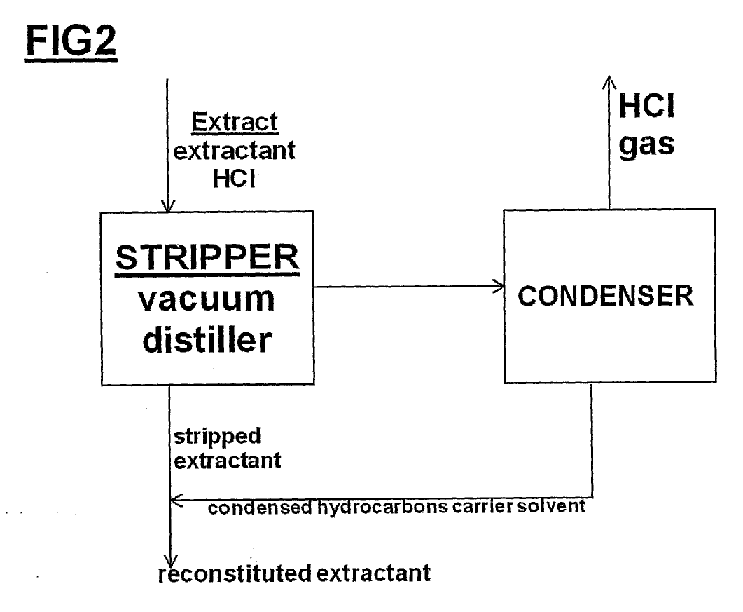

FIG. 2 is a schematic flow diagram of recovery of HCl gas using distilled hydrocarbon vapors from the carrier solvent; and

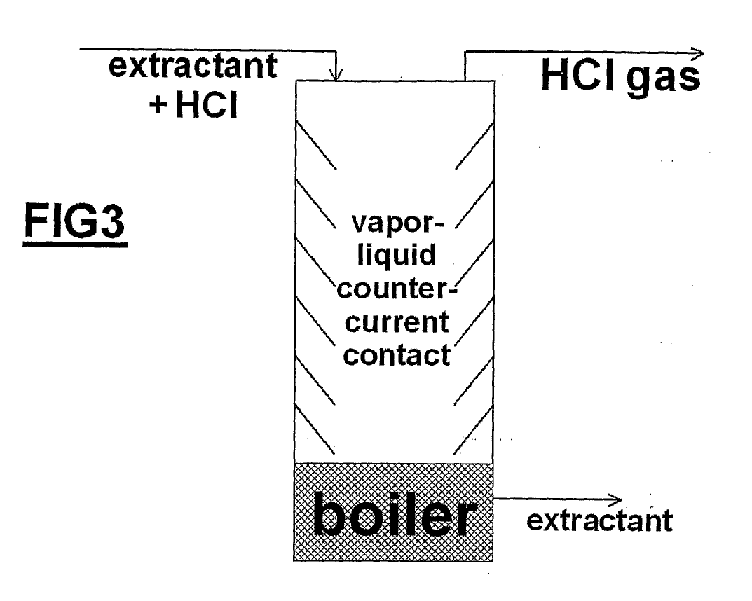

FIG. 3 is a schematic flow diagram of a system wherein the boiler, condenser and stripper are compacted into a single operation.

More specifically, referring now to FIG. 1, there is illustrated stripping by means of vapors 2 of an auxiliary hydrocarbon, which is indicated as being xylene in this particular example. The vapor is generated by boiling the xylene in boiler 4 at atmospheric pressure at 135° C. It enters the stripper 6, typically a packed column, where it meets in counter-current contact mode, an HCl-loaded extractant 8 produced in a preceding operation and preheated to 135° C. The extractant 10 is stripped off the HCl and exits the stripper at 135° C. to be cooled and returned to HCl extraction. The HCl exits with the xylene vapors 12 and enters a cooler/condenser 14. The xylene condenses and is recycled 16 to the xylene boiler and all of the HCl is recovered as water-free gas 18.

Referring now to FIG. 2 there is illustrated stripping by hydrocarbon vapor similar to FIG. 1 with the difference that no auxiliary hydrocarbon is used to generate the vapor. Instead an HCl-loaded extractant 8 produced in a preceding operation and preheated to 135° C. is introduced into a stripper 20 which includes a vacuum distiller, and wherein vapor 22 is generated by heating the HCl-loaded extractant 8 under vacuum which induces boiling and formation of vapors 22 of the hydrocarbon carrier solvent of the extractant itself. The vacuum is adjusted to secure the desired stripping temperature and the other operations are the same as described with reference to FIG. 1 with the difference that the condensed hydrocarbon carrier solvent 26 is returned to the stripped extractant 10 in order to form reconstituted extractant 28.

Referring to FIG. 3 there is illustrated an embodiment wherein the boiler 34, condenser 36 and stripper 38 are compacted into a single operation. The boiler 34 which is positioned at the bottom of the stripping column 38 keeps an HCl-free extractant boiling and generates carrier solvent vapors at the required temperature. These vapors are condensed by contact with the HCl-loaded extractant fed at the top 40 of the column 38, and the liberated HCl gas 18, of negligible solubility in this loaded feed, exits the column 38.

### EXAMPLES

### Example 1

The strong sulfonic acid LAS and the weak carboxylic acid—caproic acid (CAP)—were compared. The extent to which these acids reduce the pH of 1N NaCl from an initial pH=7.2 were 1.9 and 6.8 respectively. Two extractants were prepared by dissolving 0.1 mol of acid and 0.1 mol of base in Dodecane to obtain 100 ml of each extractant. 30 ml of each extractant was loaded with HCl by contacting with 100 ml 10% hydrochloric acid—a molar excess of about tenfold. 5 ml samples of each loaded extractant were placed on conical-bottom test tubes placed in an oil-bath kept at 170° C./173° C. and tested for stripping by sparging a stream of 30 ml/min N₂ through a capillary reaching the conical bottom of the tube for 6 min. The results are tabulated below. The tabulated stripping figures are averages of three runs.

TABLE 2

| Comparative observation | LAS:JMT | CAP:JMT |
|---|---|---|
| Molar Loading: Acid:Base:HCl | 1:1:0.23 | 1:1:0.97 |
| HCl loading after stripping | 0.09 ± 0.02 | 0.61 ± 0.2 |
| Mols HCl recovered per mol of | 0.14 | 0.36 |
| acid:base solvent | | |
| Volume of extractant per mol | Over 7 | Under 3 |
| HCl, liters | | |

The carboxylic acid obviously provides for overall improved efficiency compared with the stronger acid.

It was further established that carboxylic acids coupled with primary amines in which the alkyl chains that attach to the N-atom are of a ramified structure, form a preferred class of solvents. Example 2 is illustrative of this class.

### Comparative example 2

1 molar extractants were prepared as described in Example 1 of two acids (the strong sulfonic LAS and the weak carboxylic CAP) and of two amines with a ramified alkyl structure: JMT—a primary amine in which the alkyl chain (R) is attached to the nitrogen by a tertiary carbon R—C(CH₃)₂NH₂, and TEHA—a tertiary amine in which three identical ramified alkyl chains are attached to the nitrogen—tris(2-ethylhexylamine) (CH₃(CH₂)₃ CH(C₂H₅)CH₂)₃N.

A sample of each extractant was equilibrated with aqueous HCl at two concentration levels: 5% and 10%, and the loaded extractants thus obtained were subjected to stripping by N₂ as described in Example 1 for 20 minutes at 165° C. The results are tabulated below.

TABLE 3

| Extractants Acid:Base 1:1 molar | LAS:JMT | CAP:JMT | LAS:TEHA | CAP:TEHA |
|---|---|---|---|---|
| Molar HCl loading by 5% | 0.11 | 0.91 | 0.08 | 0.96 |
| hydrochloric acid | | | | |
| Residual HCl after stripping | 0.02 | 0.31 | 0.01 | 0.21 |
| % HCl stripped | 81.8 | 65.9 | 87.5 | 78.1 |
| Extractant per mol of recovered | 11 | 2 | 14 | 1 |
| HCl in (rounded) liters | | | | |
| Molar HCl loading by 10% | 0.23 | 1.01 | 0.11 | 1.0 |
| hydrochloric acid | | | | |
| Residual HCl after stripping | 0.04 | 0.26 | 0.005 | 0.22 |
| % HCl stripped | 82.6 | 74.3 | 95.0 | 78 |
| Extractant per mol of recovered | 5 | 1 | 2 | 1 |
| HCl in (rounded) liters | | | | |

Clearly, as expected from the teachings of co-pending PCT application, PCT/IL2008/000278, the strong acid LAS shows systematically, all else being equal, a more effective stripping than the weak acid CAP when the stripping is expressed in % HCl stripped. However, the magnitudes of stripping effectiveness for LAS and for CAP are of the same order of magnitude though their acid strengths, as measured by pKₐ, differ by several orders of magnitude. T he data also clearly indicates the advantage of the weak acid in extractant volume requirements that for low % HCl systems can reach 10 to 1. These decrease with increasing concentrations, but still are in the 2 to 1 range.

### Example 3

Stripping by N₂ and by xylene vapor were compared. The extractant was CAP:TEHA (CAP=caproic acid; TEHA=tris-(2-ethylhexylamine)) in Dodecane HCl-loaded to form an extract CAP:TEHA:HCl in the molar proportion of 1:1:1. N₂ stripping was by passing 60 ml/mn through a capillary reaching the bottom of a conical test tube containing 3 grs of the extract placed in a thermostat kept at 150° C. The stripping by xylene vapor was similar in every detail to N₂ stripping. The xylene vapor was generated by dropping xylene into a heated copper tube at the rate of 0.25 grs/min. The results of four stripping experiments for N₂ and four averaged experiments for xylene vapor are tabulated below in Table 4.

TABLE 4

| | N₂ HCl | | Xylene vapor HCl | |
|---|---|---|---|---|
| | remaining, | % | remaining, | % |
| Minutes | molar | recovered | molar | Recovered |
| 0 | 1 | 0 | 1 | 0 |
| 10 | 0.703 | 29.7 | 0.690 | 31.0 |
| 20 | 0.520 | 48.0 | 0.533 | 46.7 |
| 30 | 0.137 | 86.3 | 0.120 | 88.0 |
| 50 | 0.117 | 88.3 | 0.019 | 98.1 |

It is to be noted that while the above example was carried out with xylene, Dodecane which boils at 224° C. is not significantly evaporated at 150° C.

### Example 4

Stripping by the distillation of a hydrocarbon which constitutes the carrier solvent component of the extractant was examined for an extractant 0.8 molar in the acid:base LAS:JMT dissolved in Dodecane and loaded with HCL to the level of 0.65 molar. 10 ml of this extract were placed in a round-bottomed flask placed in a thermostatic bath maintained at 160° C. A vacuum of about 60 mm induced gentle boiling. The vapors collected in a cooled water trap. The boiling was stopped after 4 minutes; 0.8 ml of hydrocarbon collected in the trap and the HCl in the water amounted to 87% of the HCl.

### Example 5

The extractants were prepared by dissolving 0.1 mol of TEHA and 0 to 0.2 mol of OCT (octanoic acid) in Dodecane to obtain 100 gr of each extractant.

30 ml aliquots of each extractant were equilibrated with 100 ml hydrochloric acid aqueous solution. The results are presented in Tables 5-10.

TABLE 5
Equilibrium data of HCl extraction with TEHA:OCT 1:0.25
Mol/Kg in Dodecane at 27° C. and 95° C.

| | | HCl in | HCl in | |
|---|---|---|---|---|
| HCl in | HCl in | Aqueous | Extractant | |
| Aqueous | Extractant | gr/1000 gr | gr/1000 gr | Temp |
| Mol/kg | Mol/kg | H2O | extractant | ° C. |
| 1.15 | 0.82 | 44.0 | 30.9 | 27 |
| 0.56 | 0.46 | 20.9 | 17.1 | 27 |
| 0.24 | 0.089 | 8.9 | 3.3 | 27 |
| 0.117 | 0.0061 | 4.3 | 0.2 | 27 |
| 0.48 | 0.38 | 18.0 | 14.2 | 27 |
| 0.49 | 0.099 | 18.2 | 3.6 | 95 |
| 1.89 | 0.88 | 74.1 | 33.2 | 95 |
| 1.28 | 0.71 | 49.0 | 26.7 | 95 |
| 0.26 | 0.014 | 9.6 | 0.5 | 95 |

TABLE 6
Equilibrium data of HCl extraction with TEHA:OCT
1:0.25 Mol/Kg in Dodecane at 27° C.

| | | HCl in | HCl in |
|---|---|---|---|
| HCl in | HCl in | Aqueous | Extractant |
| Aqueous | Extractant | gr/1000 gr | gr/1000 gr |
| Mol/kg | Mol/kg | H2O | extractant |
| 0.039 | 0.019 | 1.4 | 0.7 |
| 0.064 | 0.050 | 2.4 | 1.8 |
| 0.154 | 0.069 | 5.7 | 2.5 |
| 0.31 | 0.24 | 11.4 | 8.7 |
| 0.42 | 0.35 | 15.8 | 13.0 |
| 0.62 | 0.56 | 23.2 | 20.9 |
| 0.78 | 0.68 | 29.5 | 25.3 |
| 1.18 | 0.87 | 45.0 | 32.7 |

TABLE 7
Equilibrium data of HCl extraction with TEHA:OCT
1:0.5 Mol/Kg in Dodecane at 27° C.

| | | HCl in | HCl in |
|---|---|---|---|
| HCl in | HCl in | Aqueous | Extractant |
| Aqueous | Extractant | gr/1000 gr | gr/1000 gr |
| Mol/kg | Mol/kg | H2O | extractant |
| 1.98 | 0.89 | 77.9 | 33.7 |
| 1.37 | 0.89 | 52.6 | 33.4 |
| 0.64 | 0.67 | 23.8 | 25.3 |
| 0.27 | 0.3 | 10.0 | 11.1 |
| 0.13 | 0.0077 | 4.8 | 0.3 |

TABLE 8
Equilibrium data of HCl extraction with
TEHA:OCT 1:1 Mol/Kg in Dodecane at 27° C.

| | | HCl in | HCl in |
|---|---|---|---|
| HCl in | HCl in | Aqueous | Extractant |
| Aqueous | Extractant | gr/1000 gr | gr/1000 gr |
| Mol/kg | Mol/kg | H2O | extractant |
| 0.113 | 0.082 | 4.1 | 3.0 |
| 0.165 | 0.34 | 6.1 | 12.6 |
| 0.46 | 0.727 | 16.9 | 27.3 |
| 1.29 | 0.91 | 49.4 | 34.3 |
| 0.83 | 0.83 | 31.1 | 31.1 |
| 2.27 | 0.94 | 90.3 | 35.4 |

TABLE 9
Equilibrium data of HCl extraction with TEHA:OCT
1:2.0 Mol/Kg in Dodecane at 25° C.

| | | HCl in | HCl in |
|---|---|---|---|
| HCl in | HCl in | Aqueous | Extractant |
| Aqueous | Extractant | gr/1000 gr | gr/1000 gr |
| Mol/kg | Mol/kg | H2O | extractant |
| 0.113 | 0.36 | 4.1 | 13 |
| 0.182 | 0.66 | 6.6 | 23.9 |
| 0.078 | 0.093 | 2.8 | 3.3 |
| 0.042 | 0.017 | 1.5 | 0.6 |

TABLE 10
Equilibrium data of HCl extraction with TEHA:OCT
1:.0 Mol/Kg in Dodecane at 25° C.

| | | HCl in | HCl in |
|---|---|---|---|
| HCl in | HCl in | Aqueous | Extractant |
| Aqueous | Extractant | gr/1000 gr | gr/1000 gr |
| Mol/kg | Mol/kg | H2O | extractant |
| 0.45 | 0.026 | 17 | 0.095 |
| 0.32 | 0.015 | 12 | 0.055 |
| 1.18 | 0.60 | 45 | 22 |
| 0.89 | 0.15 | 34 | 5.3 |
| 2.5 | 0.83 | 103 | 30.4 |

### Example 6

Extractants were prepared by dissolving 0.1 mol of TEHA and 0 to 0.1 mol of OCT (octanoic acid) in Dodecane to obtain 100 gr of each extractant. 5 ml samples of each extractant were placed in conical-bottom test tubes placed in an oil-bath kept at 170° C./173° C. The equilibrium between the HCl loaded on the extractant and the HCl in the gaseous phase was tested by bubbling a gaseous mixture of HCl and N₂ through a capillary reaching the conical bottom of the tube for 3 hours. The ratio between the N₂ and the HCl in the gas mixture and the concentration of the HCl in the extractant in equilibrium are tabulated below. The tabulated stripping figures are averages of three runs. The results are presented in Table 11

TABLE 11
The concentration of HCl in the extractant in
equilibrium with gas mixture of HCl and N₂

| Oct:TEHA in | | | N₂/HCl in the |
|---|---|---|---|
| Extractant | Temperature | Mol HCl/mol | gas phase |
| Mol/mol | (° C.) | Amine | Mol/Mol |
| 0 | 166 | 0.21 | 13.4 |
| 0 | 165 | 1.1 | 3.9 |
| 0.25 | 164 | 0.25 | 24.3 |
| 0.25 | 164 | 0.94 | 4.9 |
| 0.35 | 164 | 0.41 | 20.7 |
| 0.35 | 164 | 0.92 | 4.9 |
| 1 | 161 | 0.51 | 46.8 |
| 1 | 160 | 1.03 | 4.7 |

### Example 7

Extractant was prepared by dissolving 0.1 mol of TEHA and 0.035 mol of OCT (octanoic acid) in Dodecane to obtain 100 gr extractant. 5 gr of the extractant was contacted with 10 ml of 20% HCl aqueous solution to reach about HCl concentration in the extractant of about 1 mol/Kg.

About 0.5 gr samples of the formed loaded extractant were added into tubes. N₂ was bubbled at a rate of about 11 ml/min through the extractant at 170° C. for given durations, after which the HCl concentration in the extractant was analyzed by titration. The results are presented in Table 12

TABLE 12
HCl removal from 1M TEHA 0.35M octanoic
acid in dodecane with N₂

| Loaded extractant | | | | |
|---|---|---|---|---|
| 1M Cl | Temp | N2 | Time | HCl |
| gr | ° C. | Ml/min | min | Mol/kg |
| 0.609 | 174 | 11.8 | 10 | 0.205 |
| 0.554 | 174 | 10.9 | 20 | 0.175 |

### Example 8

Extractants were prepared by dissolving TEHA, lauric acid and caproic acidin Tetradecane. The molar ratio between lauric and caproic was 1:1 in all cases. The molar ratio between the combined acids and the amine was varied and so was the amine concentration.

Gaseous HCl+N₂ mixtures were prepared in a pressure vessel where HCl partia;l vapor pressure was in the range between zero and about 0.5 atm and the total pressure was 9.5 atm. HCl partial vapor pressure in the mixture was determined by bubbling a known amount of the gas into NaOH solution and analyzing the solution for Cl.

The gas mixture was bubbled trough the various extractants at selected temperature until an equilibrium was reached.

HCl concentration in the extractants in equilibrium was analyzed by titration. The results are presented in Table 13, where LC/T denotes the molar ratio between the combined organic acids and the amine and Z the molar ratio between the HCl in the extractant and the amine there.

TABLE 13

| | | HCl partial vapor pressure | Equilibrium HCl | |
|---|---|---|---|---|
| | Temp | in the gas mixture | in the extractant | |
| Extractant | (° C.) | mmHg | mol/kg | Z |
| LC/T 0.35 TEHA 1.0 mole/Kg | 152 | 50 | 0.62 | 0.62 |
| LC/T 0.35 TEHA 1.0 mole/Kg | 166 | 29 | 0.23 | 0.22 |
| LC/T 0.35 TEHA 1.0 mole/Kg | 164 | 127 | 1.01 | 1.01 |
| LC/T 0.25 TEHA 1.78 mole/Kg | 161 | 29 | 0.40 | 0.23 |
| LC/T 0.35 TEHA 1.78 mole/Kg | 152 | 50 | 1.25 | 0.70 |
| LC/T 0.35 TEHA 1.78 mole/Kg | 152 | 127 | 1.51 | 0.85 |
| LC/T 0.35 TEHA 1.78 mole/Kg | 158 | 65 | 0.76 | 0.43 |
| LC/T 0.35 TEHA 1.78 mole/Kg | 160 | 143 | 1.81 | 1.02 |
| LC/T 0.35 TEHA 1.78 mole/Kg | 161 | 86 | 0.82 | 0.46 |
| LC/T 0.35 TEHA 1.78 mole/Kg | 162 | 50 | 0.58 | 0.33 |
| LC/T 0.35 TEHA 1.78 mole/Kg | 162 | 127 | 1.59 | 0.89 |
| LC/T 0.35 TEHA 1.78 mole/Kg | 164 | 25 | 0.51 | 0.29 |

### Example No. 9

5 gr extractant composed of 0.8 Mol/Kg of TEHA and 0.8 Mol/Kg Lauric acid in Dodecane was loaded with 0.247 Mol/kg HCl.

2.1 gr samples of the loaded extractant were introduced into tubes. N₂ was bubbled at a rate of 30 ml/min through the extractant at 170° C. or 160° C. for different durations, after which the HCl concentrations in the extractants were analyzed by titration. The results are presented in Table 14

TABLE 14

| | Time | HCl in | HCl in |
|---|---|---|---|
| Temp | Heating | Extractant | Extractant |
| ° C. | minutes | mol/kg | mMoles |
| 170 | 0 | 0.247 | 0.521 |
| | 10 | 0.096 | 0.154 |
| | 20 | 0.033 | 0.070 |
| | 40 | 0.007 | 0.011 |
| 160 | 0 | 0.247 | 0.606 |
| | 10 | 0.218 | 0.535 |
| | 20 | 0.065 | 0.160 |
| | 40 | 0.0062 | 0.015 |

### Example No. 10

5 gr samples of extractant composed of 1.0 Mol/Kg of TEHA and 1 Mol/Kg Capric acid in Dodecane were equilibrated with 5 gr aqueous HCl solutions at 24° C. The equilibrium concentrations of HCl in the organic and in the aqueous phase are presented in Table 15.

TABLE 15

| | | | HCl in | HCl in |
|---|---|---|---|---|
| | HCl in | HCl in | Aqueous | Extractant |
| | Aqueous | Extractant | gr/1000 gr | gr/1000 gr |
| | Mol/kg | Mol/kg | H2O | extractant |
| 0 | 0.090 | 0.021 | 3.31 | 0.77 |
| 1 | 0.143 | 0.103 | 5.27 | 3.76 |
| 2 | 0.199 | 0.35 | 7.32 | 13.0 |
| 3 | 0.33 | 0.65 | 12.2 | 24.4 |
| 4 | 0.63 | 0.81 | 23.5 | 30.9 |
| 5 | 0.93 | 0.91 | 35.0 | 34.6 |
| 6 | 2.1 | 0.95 | 83.0 | 36.2 |
| 7 | 3.8 | 1.03 | 161 | 39.3 |
| 8 | 6.2 | 1.17 | 291 | 45.1 |

### Example No 11

5 gr samples of extractant composed of 0.8 Mol/Kg of TEHA and 0.8 Mol/Kg Lauric acid in Dodecane were equilibrated with 5 gr aqueous HCl solutions at 24° C. The equilibrium concentrations of HCl in the organic and in the aqueous phase are presented in Table 16.

TABLE 16

Extractant

| HCl in | HCl/ | HCl in | HCl/ |
|---|---|---|---|
| aqueous | water | Extractant | extractant |
| mol/kg | gr/kg | mol/kg | gr/kg |
| 0.223 | 8.2 | 0.13 | 4.8 |
| 1.19 | 45.4 | 0.29 | 10.5 |
| 1.63 | 63.3 | 0.52 | 19.3 |
| 2.31 | 92.1 | 0.79 | 29.6 |

1.215 gr of the extractant loaded with 0.79 mol/kg HCl was introduced into a tube. N₂ was bubbled at a rate of 30 ml/min through the extractant, which was kept at 170° C. The HCl concentration in the extractant was analyzed, at various time intervals by titration. The results are presented in Table 17

TABLE 17

| | HCl in | Amount of HCl in |
|---|---|---|
| Time | Extractant | Extractant |
| minutes | mol/kg | mMol |
| 0 | 0.79 | 0.948 |
| 10 | 0.50 | 0.513 |
| 40 | 0.39 | 0.403 |
| 60 | 0.35 | 0.359 |

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

1.-68. (canceled)

69. A process for the recovery of HCl from a dilute solution thereof, comprising:
a) bringing a dilute aqueous HCl solution into contact with a substantially water-immiscible extractant, said extractant comprising:
1) an oil soluble amine, which amine is substantially water insoluble both in free and in salt form;
2) an oil soluble weak organic acid having a pKa above 3, which acid is substantially water insoluble both in free and in salt form; and
3) a solvent for the amine and organic acid; whereupon HCl selectively transfers to said extractant to form an HCl-carrying extractant; and
b) treating said HCl-carrying extractant to obtain gaseous HCl.

70. A process according to claim 69, wherein said treating comprises heating and wherein said heating is to a temperature of up to 200° C.

71. A process according to claim 69, wherein said extractant is characterized by a pHhn of less than 3.

72. A process for the production of carbohydrates, comprising:
a) providing a lignocellulosic material comprising a polysaccharide;
b) hydrolyzing said polysaccharide in an HCl-containing hydrolysis medium to form a carbohydrate containing, dilute aqueous HCl solution;
c) bringing said dilute aqueous HCl solution into contact with a substantially water-immiscible extractant, said extractant comprising:
1) an oil-soluble amine, which amine is substantially water-insoluble, in both free and salt forms;
2) an oil soluble weak organic acid having a pKa above 3, which acid is substantially water insoluble both in free and in salt form; and
3) a solvent for the amine and organic acid; whereupon HCl selectively transfers to said extractant to form an HCl-carrying extractant and an HCl-depleted, carbohydrate-containing solution;
d) treating said HCl-carrying extractant to obtain gaseous HCl; and
e) using said gaseous HCl for hydrolysis of a polysaccharide.

73. A process according to claim 72, wherein said extractant is characterized by a pHhn of less than 3.

74. A process according claim 72, wherein said carbohydrate-containing, dilute aqueous HCl solution comprises at least one impurity and upon bringing said carbohydrate-containing dilute aqueous HCl solution in contact with said extractant, said at least one impurity transfers to said extractant to form an HCl-carrying and impurity-carrying extractant and an HCl-depleted and impurity-depleted carbohydrate-containing solution.

75. A process according to claim 69, wherein treating said HCl-carrying extractant comprises introducing a stream of an inert stripping gas comprising a hydrocarbon in vapor phase into said HCl-carrying extractant for conveying the HCl from said extractant phase and for obtaining gaseous HCl.

76. A process according to claim 72, wherein treating said HCl-carrying extractant comprises introducing a stream of an inert stripping gas comprising a hydrocarbon in vapor phase into said HCl-carrying extractant for conveying the HCl from said extractant phase and for obtaining gaseous HCl.

77. A composition comprising:
a) an oil soluble amine having a pHhn<3.5, which amine is substantially water insoluble both in free and in salt form;
b) an oil soluble weak organic acid having a pKa>3, which acid is substantially water insoluble both in free and in salt form;
c) a solvent for the amine and for the organic acid; and
d) at least one water-soluble acid selected from the group consisting of HCl, at least one non-volatile acid and combinations thereof, wherein at least one organic phase exists.

78. A composition according to claim 77, wherein said at least one water soluble acid is HCl.

79. A composition according to claim 77 further comprising water; wherein at least one organic phase and at least one aqueous phase exist and wherein said water-soluble acid is distributed between said organic phase and said aqueous phase.

80. The composition of claim 79 wherein both said organic phase and said aqueous phase comprise HCl and at least one non-volatile acid.

81. The composition of claim 79 wherein said organic phase comprises at least 0.5 mole water-soluble acid per mole of amine.

82. A composition according to claim 77 comprising:
a) an oil soluble amine having pHhn<3.5, which amine is substantially water insoluble both in free and in salt form;
b) an oil soluble weak organic acid having a pKa>3, which acid is substantially water insoluble both in free and in salt form;
c) a solvent for the amine and organic acid; and
d) at least one volatile, water soluble acid; wherein at least one organic phase and at least one vapor phase exist and wherein said volatile acid is distributed between said organic phase and said vapor phase.

83. The composition of claim 82, wherein said volatile acid is HCl.

84. The composition of claim 82 wherein the concentration of said volatile acid in said organic phase is less than 0.3 mole per mole of amine and the partial vapor pressure of said volatile acid in said vapor phase is greater than 10 mmHg.
